(19) United States
(12) Patent Application Publication
(10) Pub. No.: US 2023/0148929 A1
(43) Pub. Date: May 18, 2023

(54) BIOMEDICAL ELECTRODE, WEARABLE DEVICE, AND CLOTHING

(71) Applicant: SHINKO ELECTRIC INDUSTRIES CO., LTD., Nagano-shi (JP)

(72) Inventors: Kenichi Mori, Nagano (JP); Masayoshi Yokoo, Yamagata (JP); Kenji Seino, Yamagata (JP)

(73) Assignee: HappyJapan Inc., Yamagata-shi (JP)

(21) Appl. No.: 17/987,044

(22) Filed: Nov. 15, 2022

(30) Foreign Application Priority Data
Nov. 16, 2021 (JP) 2021-186445

(51) Int. Cl. A61B 5/27; A61B 5/00; A61B 5/256; A61B 5/389

(52) U.S. Cl. CPC A61B 5/27; A61B 5/6804; A61B 5/256; A61B 5/389

(57) ABSTRACT

A wearable device includes a biomedical electrode that can be placed on the target living subject for measurement and that detects the bioelectric potential according to the electric current coming from the target living subject for measurement; and a detection circuit that detects biological signals from the detected bioelectric potential. The biomedical electrode includes an elastic body that has a frame member having an opening and nonconductive; and includes a conductive fiber that is placed on the surface of the elastic body and that detects the bioelectric potential according to the electric current coming from the target living subject for measurement.

### CROSS-REFERENCE TO RELATED APPLICATION

This application is based upon. and claims the benefit of priority of the prior Japanese Patent Application. No. 2021-186445, filed on Nov. 16, 2021, the entire contents of which are incorporated herein by reference.

### FIELD

The embodiments discussed herein are related to a. biomedical electrode, a wearable device, and clothing.

### BACKGROUND

As a conventional wearable device; for example, a device is known that is included in the clothing worn by the target living subject for measurement, and that obtains biological signals such as heart-rate signals from the living subject who is wearing the clothing. The wearable device includes, for example, a biomedical electrode that detects the bioelectric potential from the minute electric current generated from the muscle fibers of the living subject, and a detection circuit that detects the biological signals according to the fluctuation of the bioelectric potential received from the biomedical electrode.

FIG. 17 is an explanatory diagram illustrating an example of a conventional biomedical electrode 100. As illustrated in FIG. 17, as the biomedical electrode 100 used in a wearable device, for example, a metallic biomedical electrode is known that makes contact with the skin surface of an arm A of the living subject. In order to ensure proper airflow, the biomedical electrode 100 has a ring-like metallic body. However, since the bioelectric potential detected by the biomedical electrode 100 is only minute in amount, the diameter of the biomedical electrode 100 is increased so as to increase the area of contact with the skin surface. Because of an increased area of contact, the biomedical electrode 100 becomes able to stably detect the bioelectric potential.

[Patent Literature 1] Japanese Patent Application Laid-open 2013-184024

[Non Patent Literature 1] H. K. Bhullar et. al., “Selective noninvasive electrode to study myoelectric signals”, Medical & Biological Engineering & Computing, published in November 1990

[Non Patent Literature 2] Jeroen van Vugt et al , “A convenient method to reduce crosstalk in. surface EMG”, Clinical Neurophysiology, published in April 2001

The conventional biomedical electrode 100 has a metallic body, and a large part of the skin surface of the living subject is a. curved surface. Hence, even if merely the diameter of the biomedical electrode is increased, the biomedical electrode cannot fit on the skin surface of the living subject and cannot make contact with the skin surface. As a result, the bioelectric potential cannot be detected.

### SUMMARY

According to an aspect of an embodiment, a biomedical electrode includes an elastic body that has a frame member having an opening and nonconductive; and a conductive fiber that is placed on surface of the elastic body and that detects bioelectric potential.

The object and advantages of the invention will be realized and attained by means of the elements and combinations particularly pointed out in the claims.

It is to be understood that both the foregoing general description and. the following detailed description are exemplary and explanatory and are not restrictive of the invention, as claimed.

### BRIEF DESCRIPTION OF DRAWINGS

FIG. 1 is an explanatory diagram illustrating an exemplary configuration of a wearable device according to a first embodiment;

FIG. 2 is an explanatory diagram illustrating an exemplary configuration of a detection circuit;

FIG. 3 is an explanatory diagram illustrating an example of a biomedical electrode of the wearable device according to the first embodiment;

FIG. 4A is a cross-sectional schematic view illustrating an example of the biomedical electrode when worn;

FIG. 4B is a cross-sectional schematic view illustrating an example of the biomedical electrode when worn;

FIG. 5A is an explanatory diagram illustrating an exemplary layout configuration during the normal use of the wearable device according to the first embodiment;

FIG. 5B is an explanatory diagram illustrating an exemplary issue faced in the wearable device according to the first embodiment;

FIG. 6 is an explanatory diagram illustrating an example of a wearable device according to a second embodiment;

FIG. 7 is an explanatory diagram illustrating an example of a biomedical electrode of the wearable device according to the second embodiment;

FIG. 8 is an explanatory diagram illustrating an exemplary configuration of a detection circuit;

FIG. 9A is a cross-sectional schematic view illustrating an example of the biomedical electrode when worn;

FIG. 9B is a cross-sectional schematic view illustrating an example of the biomedical electrode when worn;

FIG. 10A is an explanatory diagram illustrating an exemplary layout configuration during the normal use of the wearable device according to the second embodiment;

FIG. 10B is an. explanatory diagram. illustrating an exemplary layout configuration during the oblique use of the wearable device according to the second embodiment;

FIG. 11 is an explanatory diagram illustrating an exemplary issue faced. in the wearable device according to the second embodiment;

FIG. 12 is an explanatory diagram illustrating an exemplary issue faced in the wearable device according to the second embodiment;

FIG. 13 is an explanatory diagram illustrating an example of a biomedical electrode of a wearable device according to a third embodiment;

FIG. 14A is a cross-sectional schematic view illustrating an example of the biomedical electrode when worn;

FIG. 14B is a cross-sectional schematic view illustrating an example of the biomedical electrode when worn;

FIG. 15A is an explanatory diagram illustrating an example of the effect of the wearable device according to the third embodiment;

FIG. 15B is an explanatory diagram illustrating an example of the effect of the wearable device according to the third. embodiment;

FIG. 16 is an explanatory diagram illustrating an example of a biomedical electrode of a wearable device according to a. fourth embodiment; and

FIG. 17 is an explanatory diagram illustrating an example of a conventional biomedical electrode.

### DESCRIPTION OF EMBODIMENTS

Exemplary embodiments of a biomedical electrode, a wearable device, and clothing according to the application concerned are described. below in detail with reference to the accompanying drawings. However, the present invention is not limited by the embodiments described below.

### First Embodiment

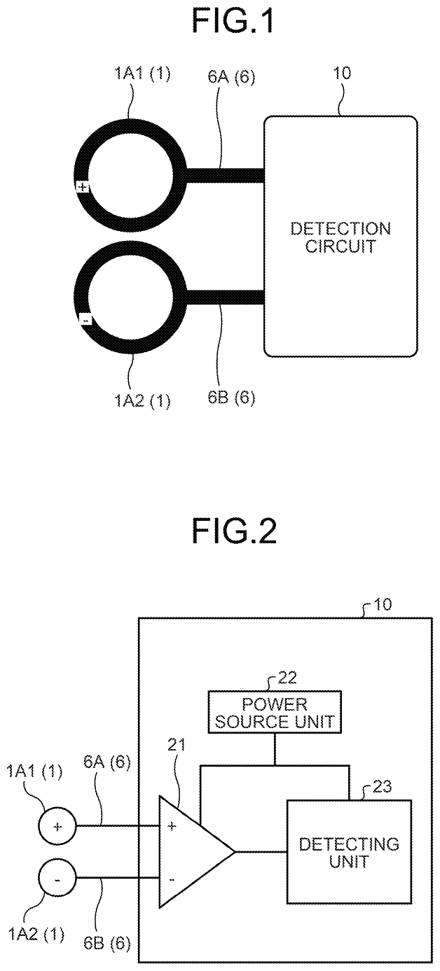

FIG. 1 is an explanatory diagram illustrating an exemplary configuration of a wearable device D according to a first embodiment. The wearable device D illustrated in FIG. 1 includes two biomedical electrodes 1, a detection. circuit 10, and wirings 6. The two biomedical electrodes 1 are made to be placeable on the skin surface of the target living subject for measurement, so that they can detect the bioelectric potential according to the electric current coming from the target living subject for measurement. The biomedical electrodes 1 include a first biomedical electrode 1A1 that detects a first-type bioelectric potential, which is the bioelectric potential of the normal phase, according to the electric current coming from the target living subject for measurement; and a second biomedical electrode 1A2 that detects a second-type bioelectric potential, which is the bioelectric potential of the reverse phase, according to the electric current coming from the target living subject for measurement.

The wirings 6 are meant for electrically connecting the biomedical electrode 1 and the detection circuit 10. The wirings 6 include a first wiring 6A and a second wiring 6B. The first wiring 6A is meant for electrically connecting the first biomedical electrode 1A1 and the detection circuit 10. The second wiring 6B is meant for electrically connecting the second biomedical electrode 1A2 and the detection circuit 10.

FIG. 2 is an explanatory diagram illustrating an exemplary configuration of the detection circuit 10. The detection circuit 10 illustrated in FIG. 2 includes a differential amplification unit 21, a power source unit 22, and a detecting unit 23. The power source unit 22 supplies electric power to the entire detection circuit 10. The differential amplification unit 21 is connected to the first wiring 6A and receives input of the first-type bioelectric potential from the first biomedical electrode 1A1, as well as is connected to the second wiring 6B and receives input of the second-type bioelectric potential from the second biomedical electrode 1A2. Moreover, the differential amplification unit 21 removes the noise component from the differential signals between the first-type bioelectric potential and the second-type bioelectric potential, and then outputs the differential signals to the detecting unit 23. Based on the differential signals, the detecting unit 23 detects the target biological signals for measurement.

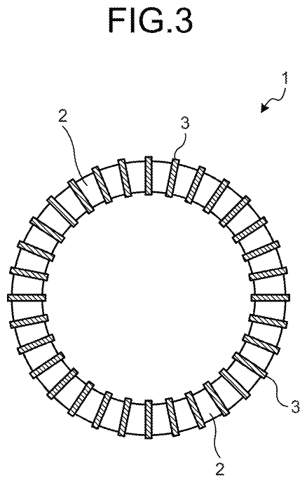

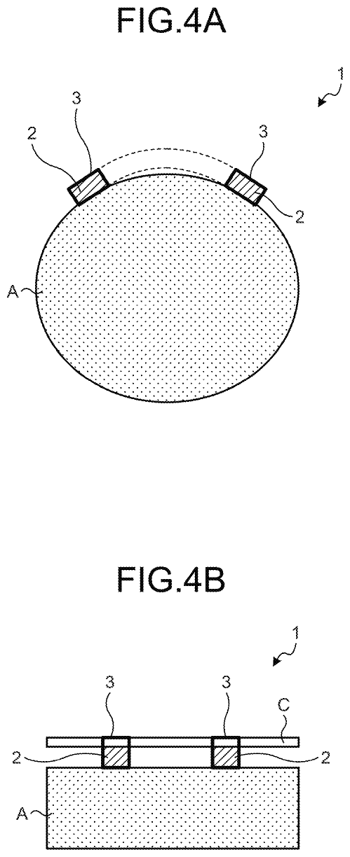

Given below is the explanation of a configuration of each biomedical electrode. FIG. 3 is an explanatory diagram illustrating an example or the biomedical electrode 1 of the wearable device D according to the first embodiment. FIG. 4A is a cross-sectional schematic view illustrating an example of the biomedical electrode 1 when worn, and FIG. 4B is a cross-sectional schematic view also illustrating an example of the biomedical electrode 1 when worn.

The biomedical electrode 1 illustrated in FIG. 3 includes an elastic body 2 that is hollow and nonconductive; and includes a conductive thread 3 that is wound around the elastic body 2, that is made of a conductive fiber, and that detects the bioelectric potential according to the electric current coming from the target living subject for measurement. The elastic body 2 is a nonconductive elastic body having a frame-like shape or a ring-like shape, such as a hollow shape having an upper face, a lower face, and a side face. The elastic body 2 has a frame member having an opening. The frame member is the frame-like shape or a ring-like shape, such as the hollow shape. In the first embodiment, the elastic body 2 is ring shaped elastic body. Moreover, the elastic body 2 has sufficient elasticity to be able to fit on the skin surface that is a curved surface.

The conductive thread 3, which is made from a conductive fiber, is a metallic thread having an uncovered and exposed surface and having conductive property. The conductive fiber is a fiber having conductive property and is made of, for example, carbon black, silver, or stainless steel.

In the biomedical electrode 1 illustrated in FIGS. 4A and 4B, clothing C and the elastic body 2 are wound and sewn together with the conductive thread 3, so that the biomedical electrode 1 is fixed to the clothing C. When the subject for measurement wears the clothing C to which the biomedical electrode 1 is sewn, the conductive thread 3 that is wound around the elastic body 2 of the biomedical electrode 1 makes contact with the skin surface of the arm A of the subject for measurement. Moreover, even when the skin surface to which the conductive thread 3 makes contact is a curved surface, the biomedical electrode 1 fits onto the skin surface due to the elasticity of the elastic body 2, and the conductive thread 3 reliably makes contact with the skin surface. As a result, the biomedical electrode 1 becomes able to stably detect the bioelectric Potential from. the conductive thread 3 that is in contact with the skin surface.

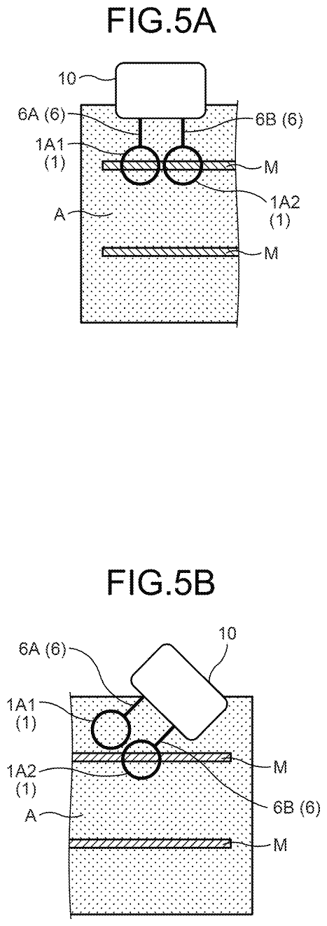

FIG. 5A is an explanatory diagram illustrating an exemplary layout configuration during the normal use of the wearable device D according to the first embodiment. In the layout configuration during the normal use as illustrated in FIG. 5A, the biomedical electrodes 1 are placed in such a way that the detection circuit 10 is oriented parallel to muscle fibers M. When the subject for measurement wears the clothing C, the first biomedical electrode 1A1 and the second biomedical electrode 1A2 that are sewn to predetermined. positions (of the arm portion) of the clothing C make contact with. the skin surface of the arm A. As a result, the first biomedical electrode 1A1 and the second biomedical electrode 1A2 make contact with the skin surface of the arm A and detect the bioelectric potential according to the minute electric current generated from the muscle fibers M.

The differential amplification unit 21 of the detection. circuit 10 receives input of the first-type bioelectric potential, which is the bioelectric potential of the normal phase, from the first biomedical electrode 1A1 via the first wiring 6A; and receives input of the second-type bioelectric potential, which is the bioelectric potential of the reverse phase, from the second biomedical electrode 1A2 via the second wiring 6B. Then, the differential amplification unit 21 outputs differential signals, which represent the difference between the first-type bioelectric potential and the second-type bioelectric potential, to the detecting unit 23. Based on the differential signals, the detecting unit 23 becomes able to detect the biological signals that are highly accurate and stable and that have the noise component removed therefrom.

In the wearable device D according to the first embodiment, from the first biomedical electrode 1A1 and the second biomedical electrode 1A2 in which the conductive thread 3 is wound around the ring-shaped elastic body 2, the first- type bioelectric potential and the second-type bioelectric potential are respectively detected; and biological signals are detected according to the differential signals between the first-type bioelectric potential and the second-type bioelectric potential. As a result, it becomes possible to obtain the biological signals that are highly accurate and stable.

in the biomedical electrode l, the conductive thread 3 is wound around the elastic body 2. Hence, even if the elastic body 2 gets deformed, disconnection attributed to the deformation of the biomedical electrode 1 can be avoided merely by increasing or reducing the winding spacing of the conductive thread 3.

Moreover, since the elastic body 2 is hollow and elastic, the biomedical electrode 1 fits onto the curved surface of the skin surface of the living subject, while ensuring proper airflow. As a result of fitting onto the skin surface that is a curved surface, the biomedical electrodes 1 can make reliable contact and stably detect the bioelectric potential.

The biomedical electrode 1 is fixed to the clothing C using the conductive thread 3 wound around the surface of the elastic body 2. Hence, the biomedical electrode 1 can be fixed to the clothing C without having to use an adhesive agent. Meanwhile, regarding the type of the clothing, as long as a nonconductive cloth is used that comes in contact with the living subject, examples of the clothing include a shirt, pants, a supporter, a bandana, underclothing, socks, gloves, a muffler, or a cap.

FIG. 5B is an explanatory diagram illustrating an exemplary issue faced in the wearable device D according to the first embodiment. If the diameter of the biomedical electrodes 1 is increased, it results in an increase in the space required for placing the first biomedical electrode 1A1 and the second biomedical electrode 1A2 on the target living subject for measurement. Thus, if the placement space cannot be secured, the first biomedical electrode 1A1, which is one of the two biomedical electrodes, cannot be placed at the target position for measurement. In that case, only one type of bioelectric potential can be obtained. As a result, in the detection circuit 10, the differential signals cannot be obtained. and, in turn, highly accurate and stable biological signals cannot be obtained.

In that regard, give below is the description of a second embodiment of a wearable device D1 in which the biomedical electrodes are downsized so that stable and highly accurate biological signals can be obtained. Herein, the identical configuration to the wearable device D according to the first embodiment is referred to by the same reference numerals, and the same configuration and the same operations are not explained again.

### Second Embodiment

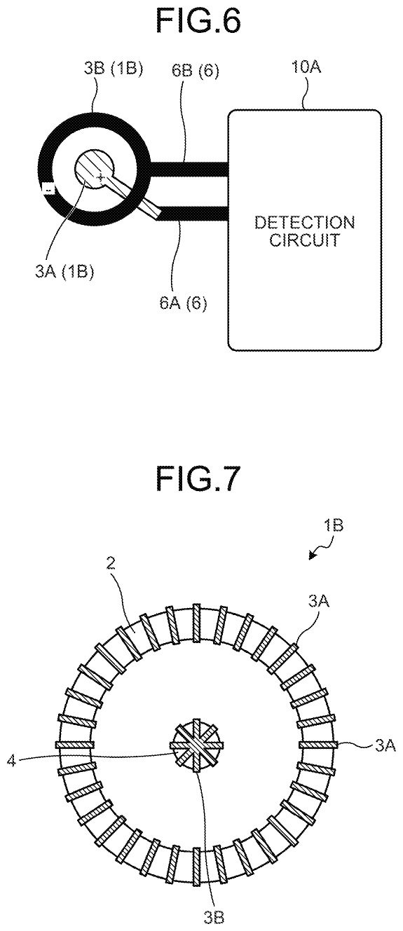

FIG. 6 is an explanatory diagram illustrating an example of the wearable device D1 according to the second embodiment. As compared to the wearable device D according to the first embodiment, the wearable device D1 illustrated in FIG. 6 differs in the way that a single biomedical electrode 1B is included that has the functions of a first biomedical electrode as well as a second biomedical electrode.

FIG. 7 is an explanatory diagram illustrating an example of the biomedical electrode 1B of the wearable device D1 according to the second embodiment. The biomedical electrode 1B illustrated in FIG. 7 includes the elastic body 2, a first conductive thread 3A, a central member 4, and a second conductive thread 3B.

The elastic body 2 is ring-shaped nonconductive elastic body. The first conductive thread 3A is wound around the elastic body 2 and represents a first biomedical electrode that detects the first-type bioelectric potential according to the electric current coming from the target living subject for measurement.

The central member 4 is a nonconductive member placed on the round core inside the ring of the elastic body 2. The central member is also an elastic body having elasticity. The second conductive thread 3B is wound around the central member 4, and represents a second biomedical electrode that detects the second-type bioelectric potential, which is the bioelectric potential of the reverse phase, according to the electric current coming from the target living subject for measurement.

The wirings 6 are meant for electrically connecting the biomedical electrode 1B and a detection circuit 10A. The wirings 6 include the first wiring 3A and the second wiring 6B. The first wring 6A is meant for electrically connecting the first conductive thread 3A and the detection circuit 10. The second wiring 6B is meant for electrically connecting the second conductive thread 3B and the detection circuit 10.

Thus, the single biomedical electrode 1B includes a first biomedical electrode in. the form of the first conductive thread 3A and includes a second biomedical electrode in the form of the second conductive thread 3B. Hence, as compared to the first biomedical electrode 1A1 and the second. biomedical electrode 1A2 according to the first embodiment, the main body of the biomedical electrode 1B can be downsized. As a result, the placement space for the biomedical electrode 1B can be reduced.

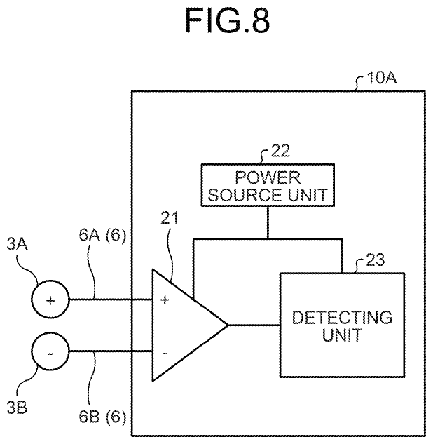

FIG. 8 is an explanatory diagram illustrating an exemplary can figuration of the detection circuit 10A. The detection circuit 10A illustrated in FIG. 8 includes the differential amplification unit 21, the power source unit 22, and the detecting unit 23. The power source unit 22 supplies electric power to the entire detection circuit 10A. The differential amplification unit 21 is connected to the first wiring 6A and receives input of the first-type bioelectric potential from the first conductive thread 3A as well as is connected to the second wiring 6B and receives input of the second-type bioelectric potential from the second conductive thread 3B. Moreover, the differential amplification unit 21 removes the noise component from the differential signals between the first-type bioelectric potential and the second-type bioelectric potential, and then outputs the differential signals to the detecting unit 23. Based on the differential signals, the detecting unit 23 detects the target biological signals for measurement.

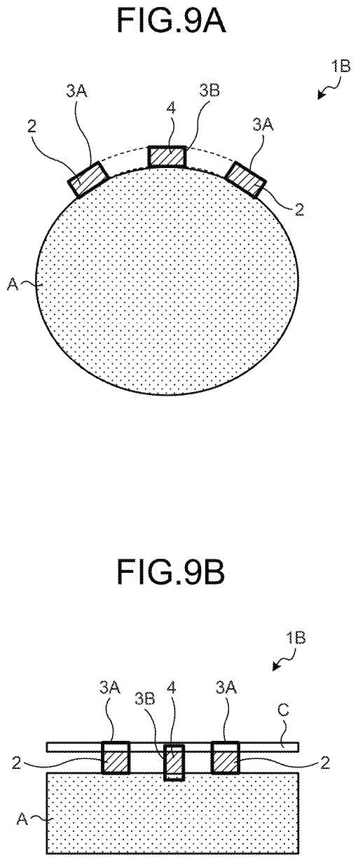

FIG. 9A is a cross-sectional schematic view illustrating an example of the biomedical electrode 1B when worn, and FIG. 9B is a cross-sectional schematic view also illustrating an example of the biomedical electrode 1 when worn. In the biomedical electrode 1B illustrated in FIGS. 9A and 9B, the clothing C and the elastic body 2 are wound and sewn together with the first conductive thread 3A, and the clothing C and the central member 4 are wound and sewn together with the second conductive thread 3B. With that, the biomedical electrode 1B is fixed to the clothing C. When the subject for measurement wears the clothing C to which the biomedical electrode 1B is sewn, the first conductive thread 3A and the second conductive thread 3B make contact with the skin surface of the arm A of the subject for measurement. Moreover, even when the skin surface to which the first conductive thread 3A and the second conductive thread 3B make contact is a curved surface, the biomedical electrode 1B fits onto the skin surface due to the elasticity of the elastic body 2, and the first conductive thread 3A and the second conductive thread 3B reliably make contact with the skin surface. As a result, the biomedical electrode 1B becomes able to stably detect the bioelectric potential from the first conductive thread 3A and the second conductive thread 3B that are in contact with the skin surface.

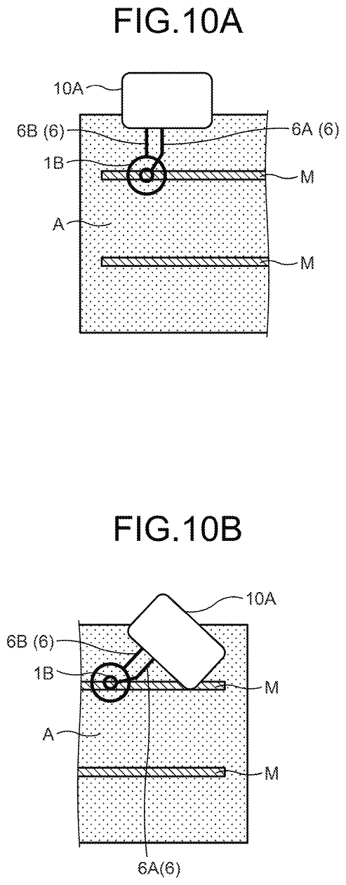

FIG. 10A is an explanatory diagram illustrating an exemplary layout configuration during the normal use of the wearable device D1 according to the second embodiment. In the layout configuration during the normal use as illustrated in FIG. 10A, the biomedical electrode 1B is placed in such a way that the detection circuit 10A is oriented parallel to the muscle fibers M. When the subject for measurement wears the clothing C, the first conductive thread 3A and the second conductive thread 3B of the biomedical electrode 1B, which are sewn to predetermined positions (of the arm portion)) of the clothing C, make contact with the skin surface of the arm A. As a result, the biomedical electrode 1B makes contact with the skin surface of the arm A and, via the first conductive thread 3A and the second conductive thread 3B, detects the bioelectric potential from the minute electric current generated from the muscle fibers M.

The differential amplification unit 21 of the detection circuit 10A receives input of the first-type bioelectric potential, which is the bioelectric potential of the normal phase, from the first conductive thread 3A via the first wiring 6A; and receives input of the second-type bioelectric potential, which is the bioelectric potential of the reverse phase, from the second conductive thread 3B via the second wring 6B. Then, the differential amplification unit 21 outputs differential signals, which represent the difference between the first-type bioelectric potential and the second-type bioelectric potential, to the detecting unit 23. Based on the differential signals, the detecting unit 23 becomes able to detect the biological signals that are highly accurate and stable and that have the noise component removed there from.

FIG. 10B is an explanatory diagram illustrating an exemplary layout configuration during the oblique use of the wearable device D1 according to the second embodiment. In the layout configuration during the oblique use as illustrated in FIG. 10B, the biomedical electrode 1B is placed in such a way that the detection circuit 10A is oriented oblique to the muscle fibers M. In the case of the oblique use too, the single biomedical electrode 1B is able to detect the first-type bioelectric potential and the second-type bioelectric potential. The differential amplification unit 21 of the detection circuit 10A receives input of the first-type bioelectric potential, which is the bioelectric potential of the normal phase, from the first conductive thread 3A via the first ring 6A; and receives input of the second-type bioelectric potential, which is the bioelectric potential of the reverse phase, from the second conductive thread 3B via the second wiring 6B. Then, the differential amplification unit 21 outputs differential signals, which represent the difference between the first-type bioelectric potential and the second-type bioelectric potential, to the detecting unit 23. Based on the differential signals, the detecting unit 23 becomes able to detect the biological signals that are highly accurate and stable and that have the noise component removed therefrom. Thus, also in the case of the oblique use in which a smaller placement space is required, the single biomedical electrode 1B can detect the first-type bioelectric potential and the second-type bioelectric potential.

In the biomedical electrode 1B of the wearable device D1 according to the second embodiment, the first conductive thread 3A is wound around the ring-shaped elastic body 2; the central member 4 is placed inside the ring of the elastic body 2; and the second conductive thread 3B is wound around the central member 4. The biomedical electrode 1B detects the first-type bioelectric potential from the first conductive thread 3A and detects the second-type bioelectric potential from the second conductive thread 3B. Thus, the first-type bioelectric potential and the second-type bioelectric potential can be obtained by a single biomedical electrode 1B, thereby enabling downsizing thereof. Moreover, as compared to the first embodiment, it becomes possible to reduce the space required for placing the biomedical electrode 1B.

Meanwhile, the central member 4 according to the second embodiment can be made from the identical material to the material of the elastic body 2 or can be made from a different material than the material of the elastic body 2. Thus, the material can be appropriately changed.

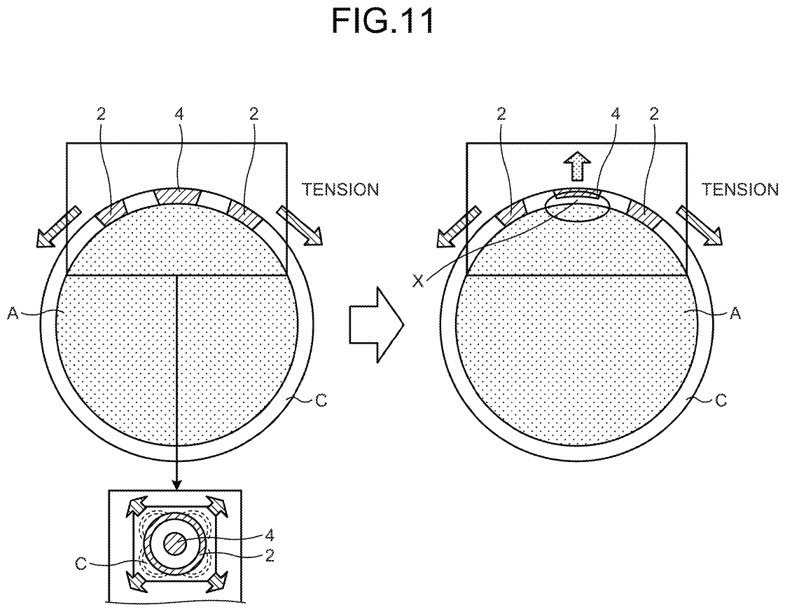

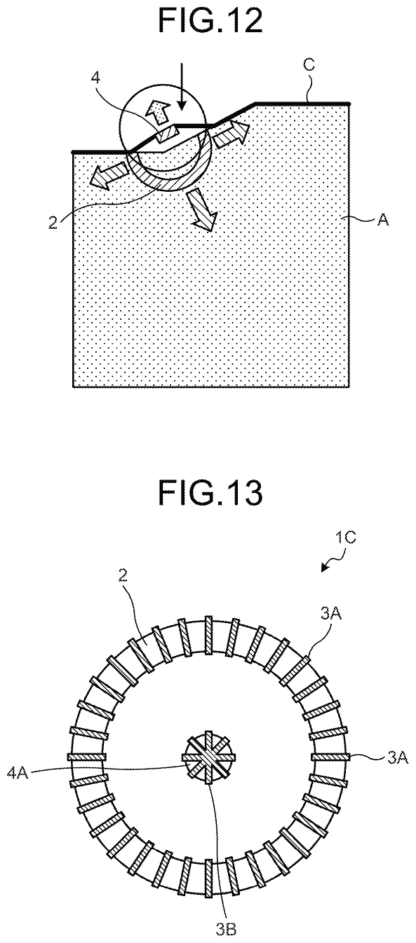

FIGS. 11 and 12 are explanatory diagrams illustrating an exemplary issue faced in the wearable device D1 according to the second embodiment. In the wearable device D1, when the subject for measurement wears the clothing C to which the biomedical electrode 1B is sewn, as illustrated in FIG. 11, the first conductive thread 3A wound around the elastic body 2 and the second conductive thread 3B wound around the central member 4 in the biomedical electrode 1B make contact with the skin surface of the arm A. However, when the clothing C gets stretched after being worn by the subject for measurement, a tension is created in the outer periphery direction of the biomedical electrode 1B. Because of the tension, it is possible to think that the central member 4 of the biomedical electrode 1B comes off and the second conductive thread 3B cannot remain in contact with the skin. surface X. In that case, the detection circuit 10A can no more obtain the second-type bioelectric potential from the second conductive thread 3B that is wound around the central member 4. Thus, in the wearable device D1, since the second-type bioelectric potential cannot be obtained, the differential signals cannot be obtained and, in turn, stable biological signals cannot be obtained. That results in a decline in the signal quality.

In that regard, given below is the description of a third embodiment of a wearable device D2 that enables avoiding the situation in which the central member 4 in the biomedical electrode 1B comes off the skin surface. Herein, the identical configuration to the wearable device D1 according to the second embodiment is referred to by the same reference numerals, and the same configuration and the same operations are not explained again.

### Third Embodiment

FIG. 13 is an explanatory diagram illustrating an example of a biomedical electrode 1C of the wearable device D2 according to the third embodiment. As compared to the wearable device D1 according to the second embodiment, the wearable device D2 according to the third embodiment differs in the way that a central member 4A in the biomedical electrode 1C has more thickness than the thickness of the elastic body 2. For example, the thickness of the central member 4A is set to be approximately twice the thickness of the elastic body 2. The central member 4A includes a first partitioned member 4A1 and a second partitioned member 4A2. Herein, in the central member 4A, the first partitioned member 4A1 and the second partitioned member 4A2 are separate members. Alternatively, the first partitioned member 4A1 and the second partitioned member 4A2 can be integrated. Thus, the configuration can be appropriately changed.

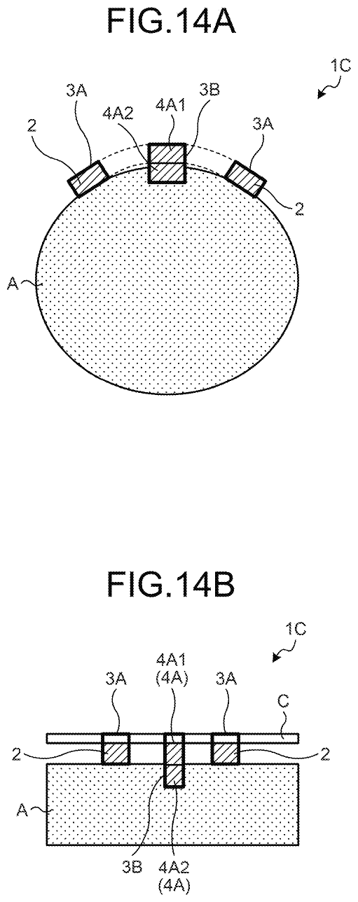

FIG. 14A is a cross--sectional schematic view illustrating an example of the biomedical electrode 1C when worn, and FIG. 14B is a cross-sectional schematic view also illustrating an example of the biomedical electrode 1C when worn. In the biomedical electrode 1C illustrated in FIGS. 14A and 14B, the clothing C and the elastic body 2 are wound and sewn together with the first conductive thread and the clothing C and the central member 4A are wound and sewn together with the second conductive thread 3B. With that, the biomedical electrode 1C is fixed to the clothing C. When the subject for measurement wears the clothing C to which the biomedical electrode 1C is sewn, the first conductive thread 3A and the second conductive read 3B reliably make contact with the skin surface of the arm A of the subject for measurement. Moreover, even when the skin surface to which the first conductive thread 3A and the second conductive thread 3B make contact is a curved surface, the biomedical electrode 1C fits onto the skin surface due to the elasticity of the elastic body 2, and the first conductive thread 3A and the second conductive thread 3B reliably make contact with the skin surface. As a result, the biomedical electrode 1C becomes able to stably detect the bioelectric potential from the first conductive thread 3A and the second conductive thread 3B that are in contact with the skin surface.

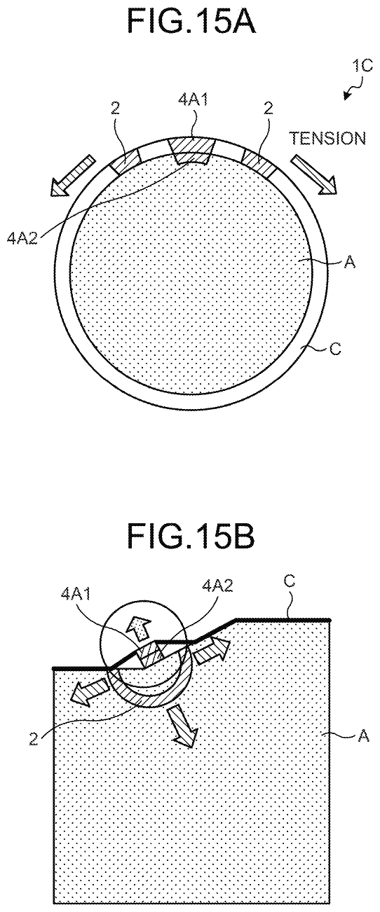

FIGS. 15A and 15B are explanatory diagrams illustrating an example of the effect of the wearable device D2 according to the third embodiment. The central member 4A is made to have a greater thickness as compared to the thickness of the elastic body 2. Hence, the central member 4A does not come off in the upward direction, and the second conductive thread 3B that is wound around the central member 4A reliably makes contact with the skin surface of the arm A. Then, via the first conductive thread 3A and the second conductive thread 3B, the wearable device D2 becomes able to stably detect the bioelectric potential from the minute electric current generated from the muscle fibers M of the arm A.

The differential amplification unit 21 of the detection circuit 10A receives input of the first-type bioelectric potential, which is the bioelectric potential of the normal phase, from the first conductive thread 3A via the first wiring 6A; and receives input of the second-type bioelectric potential, which is the bioelectric potential of the reverse phase, from the second conductive thread 3B via the second wiring 6B. Then, the differential amplification unit 21 outputs differential signals, which represent the difference between the first-type bioelectric potential and the second-type bioelectric potential, to the detecting unit 23. Based on the differential signals, the detecting unit 23 becomes able to detect the biological signals that are highly accurate and stable and that have the noise component removed therefrom.

In the biomedical electrode 1C of the wearable device D2 according to the third embodiment, at the time of placing the central member 4A inside the ring of the ring-shaped elastic body 2, the thickness of the central member 4A is kept to be greater than the thickness of the elastic body 2. For example, when the wearable device D2 is worn on the arm A, the ring-shaped elastic body 2 is pulled in the outer periphery direction. Thus, even. if the central member 4A inside the ring of the elastic body 2 comes off in the upward direction, the tension exerted on the clothing C presses the central member 4A onto the skin sulfate. As a result, the central member 4A reliably makes contact with the skin surface of the subject for measurement. That results in stabilizing the contact between the second conductive thread 3B and the skin surface of the arm A of the subject for measurement, thereby enabling obtaining stable differential signals.

Meanwhile, in the biomedical electrode 1B of the wearable device D1 according to the second embodiment, the elastic body 2 and the central member 4 are illustrated to be independent members. However, that is not the only possible case. In that regard, given below is the description of a fourth embodiment. Herein, the identical configuration to the wearable device D1 according to the second embodiment is referred to by the same reference numerals, and the same configuration and the same operations are not explained again.

### Fourth Embodiment

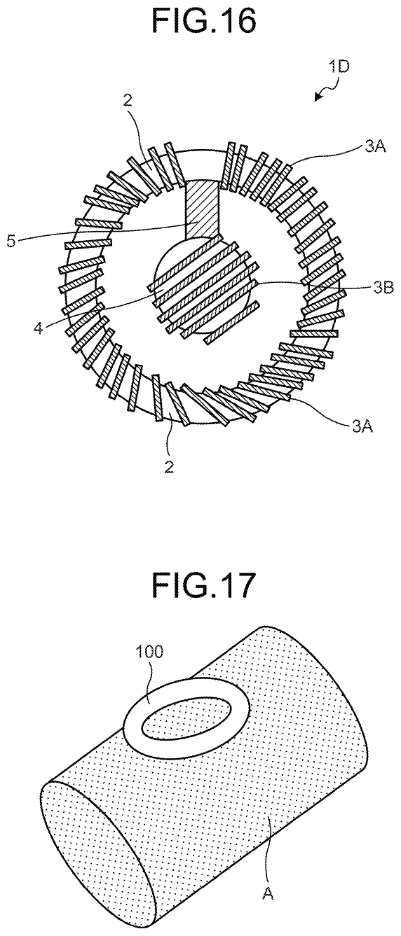

FIG. 16 is an explanatory diagram illustrating an example of a biomedical electrode 1D of a wearable device D3 according to the fourth embodiment. As compared to the wearable device D1 according to the second embodiment, the wearable device D3 a cording to the fourth. embodiment differs in the way that nonconductive joining section 5 is provided for joining the elastic body 2 and the central member 4 of the biomedical electrode 1D, and that the central member 4 is fixed inside the ring of the elastic body 2 using the joining section 5. Thus, the elastic body 2, the central member 4, and the joining section 5 are formed in an integrated manner.

When the subject for measurement wears the clothing C, the first conductive thread 3A and the second conductive thread 3B of the biomedical electrode 1D that are sewn to pre determined positions (of the arm portion) of the clothing C make contact with the skin surface of the arm A. Then, via the first conductive thread 3A and the second conductive thread 3B, the biomedical electrode 1D detects the bioelectric potential from the minute electric current generated from the muscle fibers M.

The differential amplification unit 21 of the detection circuit 10A receives input of the first-type bioelectric potential, which is the bioelectric potential of the normal phase, from the first conductive thread 3A via the first wiring 6A; and receives input of the second-type bioelectric potential, which is the bioelectric potential potential of the reverse phase, from the second conductive thread 3B via the second wiring 6B. Then, the differential amplification unit 21 outputs differential signals, which represent the difference between the first-type bioelectric potential and the second-type bioelectric potential, to the detecting unit 23. Based on the differential signals, the detecting unit 23 becomes able to detect the biological signals that are highly accurate and stable and that have the noise component removed therefrom.

In the biomedical electrode 1D of the wearable device D3 according to the fourth embodiment, the first conductive thread 3A is wound around the ring-shaped elastic body 2; and the second conductive thread 3B is wound around the central member 4 that is joined to the elastic body 2 using the joining section 5. The biomedical electrode 1D detects the first-type bioelectric potential from the first conductive thread 3A and detects the second-type bioelectric potential from the second conductive thread 3B. Thus, the first-type bioelectric potential and the second-type bioelectric potential can be obtained by a single biomedical electrode 1D, thereby enabling downsizing thereof. Moreover, as compared to the first embodiment, it becomes possible to reduce the space required for placing the biomedical electrode 1D.

Meanwhile, for explanatory convenience, the elastic body 2, the central member 4, and the joining section 5 are formed in an integrated manner. Alternatively, they can be formed separately. Thus, the configuration can be appropriately changed.

Regarding the biomedical electrode 1D, the clothing C is separately wound and sewn with the first conductive thread 3A and with the second conductive thread 3B. Alternatively, the clothing C can be wound and sewn either with the first conductive thread 3A or with the second conductive thread 3B. Thus, the configuration can be appropriately changed.

Moreover, in the biomedical electrode 1D, a single joining section 5 is used to join the elastic body 2 and the central member 4. Alternatively, a plurality of joining sections 5 can be used to join the elastic body 2 and the central member 4. Thus, the configuration can be appropriately changed.

Meanwhile, although the explanation here is given for the case in which the elastic body 2 is sewn to the clothing C using the conductive thread 3, it is also possible to sew the elastic body 2 to the clothing C using some other type of thread.

Moreover, the explanation is given about the case in which the elastic body 2 and the clothing C are wound together with the first conductive thread 3A, and thus the elastic body 2 is sewn to the clothing C. Alternatively the first conductive thread 3A that is wound around the elastic body 2 can be sewn to the clothing C. Thus, the configuration can be appropriately changed. Moreover, the elastic body 2 can be sewn to the clothing C using some other type of thread.

Furthermore, the explanation is given about the case in which the central member 4 and the clothing C are wound together with the second conductive thread 3B, and thus the central member 4 is sewn to the clothing C. Alternatively, the second conductive thread 3B that is wound around the central member 4 can be sewn to the clothing C. Thus, the configuration can be appropriately changed. Moreover, the central member 4 can be sewn to the clothing C using some other type of thread.

Furthermore, the explanation is given about the case in which the central member 4 is placed on the round core present inside the ring of the elastic body 2. However, the central member 4 is not limited to be placed on the round core. That is, as long as the central member 4 is placed inside the ring of the elastic body 2, it serves the purpose. Thus, the configuration can be appropriately changed.

Moreover, the explanation is given about the example in which the conductive thread 3 is wound around the surface of the elastic body 2. However, it is not limited to use the conductive thread 3 for winding. Alternatively, the surface of the elastic body 2 can be coated using a conductive fiber. Thus, the configuration. can be appropriately changed. Moreover, the conductive fiber either can have the conductive property and the aeration property or can be a non-aerating fiber.

Meanwhile, the explanation is given about the case in which two biomedical electrodes 1A1 and 1A2 are used in the wearable device D according to the first embodiment. Alternatively, only a single biomedical electrode 1 can be used. Thus, the configuration can be appropriately changed.

According to an aspect of the biomedical electrode disclosed in the application concerned, the biomedical electrode can be fit onto a skin surface, which is a curved surface, using the elastic of the elastic body. With that, the bioelectric potential can be detected in a stable manner.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A biomedical electrode comprising:
an elastic body that has a frame member having an opening and nonconductive; and
a conductive fiber that is placed on surface of the elastic body and that detects bioelectric potential.

2. The biomedical electrode according to claim 1, wherein the conductive fiber is wound around the surface of the elastic body.

3. The biomedical electrode according to claim 1, further including:
a central member that is nonconductive and that is placed inside the frame member of the elastic body; and
other conductive fiber that is placed on surface of the central member.

4. The biomedical electrode according to claim 3, wherein thickness of the central member is greater than thickness of the elastic body.

5. The biomedical electrode according to claim 3, further including a joining section that is nonconductive and that joins the elastic body and the central member.

6. The biomedical electrode according to claim 3, wherein
the conductive fiber detects the bioelectric potential according to electric current coming from target living subject of measurement, and
the other conductive fiber detects bioelectric potential of reverse phase according to electric current coming from the target living subject for measurement.

7. A wearable device comprising:
a biomedical electrode that is placeable on target living subject for measurement and that detects bioelectric potential according to electric current coming from the target living subject for measurement; and
a detection circuit that detects biological signal from the detected bioelectric potential, wherein
the biomedical electrode includes
an elastic body that has a frame member having an opening and nonconductive, and
a conductive fiber that is placed on surface of the elastic body and that detects the bioelectric potential according to electric current coming from the target living subject for measurement.

8. Clothing comprising:
a biomedical electrode that includes
an elastic body that has a frame member having an opening and nonconductive, and
a conductive fiber that is placed on surface of the elastic body and that detects bioelectric potential; and
a fabric to which the elastic body is wound using the conductive fiber, so that the biomedical electrode is fixed to the fabric.

9. The clothing according to claim 8, further including:
a central member that is nonconductive and that is placed inside the frame member of the elastic body; and
other conductive fiber that is placed on surface of the central member.

10. The clothing according to claim 9, wherein thickness of the central member is greater than thickness of the elastic body.
